(19) United States
(12) Patent Application Publication
(10) Pub. No.: US 2007/0240988 A1
(43) Pub. Date: Oct. 18, 2007

(54) Method for controlling concentration of electrophoresis solution of carbon nano tube

(75) Inventors: Kuei-Wen Cheng, Guanyin Township (TW); Shie-Heng Lee, Guanyin Township (TW)

Correspondence Address: HDSL, 4331 STEVENS BATTLE LANE, FAIRFAX, VA, 22033, US

(73) Assignee: Teco Electric & Machinery Co., Ltd.

(21) Appl. No.: 11/404,790

(22) Filed: Apr. 17, 2006

(51) Int. Cl. C07K 1/26

(52) U.S. Cl. 204/450

(57) ABSTRACT

A method for controlling concentration of electrophoresis solution of field emission display cathode substrate carbon nano tubes includes: (1) preparing an index electrophoresis solution and setting the electrophoresis conditions, wherein the concentration is 0.025% for magnesium nitrate and 0.0125% for the carbon nano tube of ethanol as an index solution; a fixed electric field 10 volt/millimeter is applied to the electrodes; (2) performing electrophoresis for the index solution and expressing the time t of current I per unit time at a preliminary stage as a function of t to establish Ii=Ii(t); (3) continuing to establish the function Ib=Ib(t); (4) comparing the ratio of a linear term and a constant of the Ib(t) and Ii(t); if the difference of the coefficients of the constant and linear term exceeds 5%, then (5) computing the charger concentration and resupplying carbon nano tubes and chargers, so that the batch solution approaches the index solution.

### BACKGROUND OF THE INVENTION

1. Field of the Invention

The present invention relates to a method for manufacturing a field emission display, and more particularly to a method for manufacturing a field emission display cathode plate carbon nano tube electron emitter.

2. Description of Prior Art

At present, electronic products are generally developed in a light, thin, short, and compact design, but the traditional cathode ray tube display is large and inconvenient-to-transport and occupies much space. To flatten the traditional cathode ray tube, a carbon nano tube field emission display (CNT-FED) with a low-pass electric field and a high emissive density is provided. The electric field activated cathode electrons are used to collide the phosphors of an anode. A field emission display several tens of active cold emitters, and the cathode ray tube uses a single electronic gun to emit electron beams and requires a deflection yoke for controlling the direction of the electrons, and thus the field emission display can achieve the effect of saving more area than the cathode ray tube. Therefore, the image quality of the field emission display is similar to that of the cathode ray tube, and has the power-saving and lightweight advantages.

The carbon nano tube field emission display uses the carbon nano tube for an electron emitter, and thus its cathode substrate includes carbon nano tubes. There are many ways of producing carbon nano tubes on the cathode substrate, and these methods include a chemical vapor deposition, or a photosensitive carbon nano tube solution pattern manufactured on the cathode conducting layer in each pixel, or a carbon nano tube solution manufactured with masking. In the structure of an electron emitter of a triode field emission display, the foregoing methods for implanting the carbon nano tubes onto a triode cathode structure incur a very high cost If it is necessary to manufacture a large electron emitter, it will be difficult to achieve the evenness of the cathode substrate. The electrophoresis deposition (EPD) can solve the large size and high cost problems. U.S. Pat. Publication No. 2003/0102222 discloses a method of using carbon nano tubes to prepare alcohol suspension as an electrophoresis solution, and the magnesium, lanthanum, yttrium, or aluminum ion salts are used as a charger, and the cathode substrate that requires a deposition is connected to an electrode and placed in an electrophoresis solution, and a DC or AC voltage is added to the electrodes to form an electric field in the solution, and the ions of the charger are attached onto the carbon nano tube powder. Since the ions are driven by the electric field of the electrophoresis to deposit the carbon nano tubes onto the electrodes, therefore the deposited carbon nano tube pattern is formed on the cathode substrate electrode.

Since the electrophoresis deposition can deposit the carbon nano tube pattern on the electrode layer and avoid the limitations of the triode emission display on the cathode structure, therefore the electrophoresis deposition is used extensively for the production of carbon nano tubes of the cathode substrate. During the process of the electrophoresis deposition, the charger that requires the deposited carbon nano tubes is gradually deposited onto the electrode, so that the concentrations of charger ions and carbon nano tubes in the solution will drop gradually, and the chargers will move to produce a change of voltage current of the electrophoresis, so as to lower the deposition rate of carbon nano tubes.

Therefore, the electrophoresis technology usually goes with the setup of a power supply, and a specific voltage or a specific current is adopted for the electrophoresis process. After a period of time, an electrophoresis deposition is produced on the surface of the electrode substrate. However, the charger ions and carbon nano tubes in the electrophoresis solution are deposited gradually, such that the solutes (chargers and carbon nano tubes) in the solution are deposited To overcome the effect caused by the change of the solute concentration, the value of the power supply or a compensated solute is adopted for the correction. The former increases the voltage, current or the electrophoresis time, and the later resupplies the solute or changes the solution.

Regardless of correcting the voltage or the current or compensating the solute, these changes still create some problems for a continuous operating process. As to the correction of the voltage or current, if the charger concentration drops to maintain a specific quantity of the electrophoresis deposition, it is necessary to increase the voltage or current. However, the ethanol solution is inflammable, and thus the increase of current may cause a burning easily. If water is used as a solvent, the water produced by the electrolysis is unfavorable for the deposition. Further, if the electrophoresis time is increased, an uneven deposition or polarization will occur easily, and thus the electrophoresis preferably achieves the deposition within the shortest possible time.

On the other hand, if we want to maintain the concentration of the reduced chargers or carbon nano tubes during the electrophoresis deposition, it is necessary to have an assistant determining mechanism to obtain the optimal resupply timing. The prior art removes the cathode substrate after 10 minutes of the electrophoresis during the process of preparing the cathode substrate, and several times of batch processing are performed to deposit an even effect (wherein the prior art carries out the batch processing for three times to achieve an even deposition effect). Therefore, the prior art checks the deposition result of the cathode substrate after the adjustment of the solute concentration to determine the concentration, and thus wasting time and unable to satisfy the mass production requirement for a continuous electrophoresis deposition.

### SUMMARY OF THE INVENTION

In view of the foregoing shortcomings of the prior art, the inventor of the present invention based on years of experience in the related industry to conduct experiments and modifications, and finally designed a mechanism for determining the concentration of an electrophoresis solution to maintain the solute concentration of the electrophoresis solution, so as to continue the electrophoresis deposition reaction and overcome the shortcomings of the prior art.

Therefore, the present invention is to overcome the shortcomings of the prior art by providing a method for determining the solute concentration of an electrophoresis solution to establish a method for producing an electrophoresis deposition of a field emission display cathode electron emitter. The method provides a mechanism for determining the concentration of an electrophoresis solution and timely resupply chargers and carbon nano tubes in the electrophoresis solution to continue the electrophoresis deposition. In the electrophoresis deposition process, the change of voltage or current relates to the concentration of the chargers in the solution, and thus the present invention uses the relation between the current and time to determine whether or not the concentration of the solution can still deposit good carbon nano tubes onto the cathode substrate.

In the electrophoresis process, the change of voltage or current is directly proportional to the concentration of the chargers in the solution. The present invention uses this characteristic to keep recording the change of current as a basis for resupplying chargers and carbon nano tubes in the electrophoresis process, such that the electrophoresis deposition can be completed within a controllable time to satisfy the mass production requirements. In addition, this method can carry out the patch electrophoresis continuously to produce electron emitters and predict the change of ion concentration of the solution for an appropriate adjustment to maintain the concentration of the electrophoresis solution within a specific range and carry out the electrophoresis continuously.

Therefore, the control method of the invention comprises the steps of:

(1) preparing an index electrophoresis solution and setting the electrophoresis conditions, wherein the concentration is 0.025% for magnesium nitrate and 0.0125% for the carbon nano tube of ethanol as an index solution; a fixed electric field 10 volt/millimeter is applied to the electrodes; and the duty factor is ⅕(on/total); and the voltage frequency is 250 Hz; (2) performing an electrophoresis for the index solution and obtaining the values of the current per unit time at the preliminary stage and the time and using a regression method to express the current in terms of a function of time to establish Ii=Ii(t), and the unit time adopted in this preferred embodiment is equal to 60 seconds, and the current is recorded once very five seconds; (3) continuing the batch electrophoresis of the solution and similarly continuing to establish the function Ib=Ib(t) of the electrophoresis solution; (4) comparing the ratio of a linear term and a constant term of the Ib(t) and Ii(t) to predict the limitations of using the ion concentration in the solution; if the difference of the ratios of the coefficients of a linear term and a constant term exceeds 5%, then (5) computing the charger concentration of the batch solution and resupplying carbon nano tubes and chargers, so that the batch solution approaches the index solution to facilitate the electrophoresis for the next time.

### BRIEF DESCRIPTION OF DRAWINGS

The features of the invention believed to be novel are set forth with particularity in the appended claims. The invention itself however may be best understood by reference to the following detailed description of the invention, which describes certain exemplary embodiments of the invention, taken in conjunction with the accompanying drawings in which:

FIG. 1 is a table of experiment data of the present invention;

FIG. 2 is a curve of x-y relation of experiment data of the present invention;

FIG. 3 is another table of experiment data of the present invention;

FIG. 4 is a curve of x-y relation of experiment data as depicted in FIG. 3; and

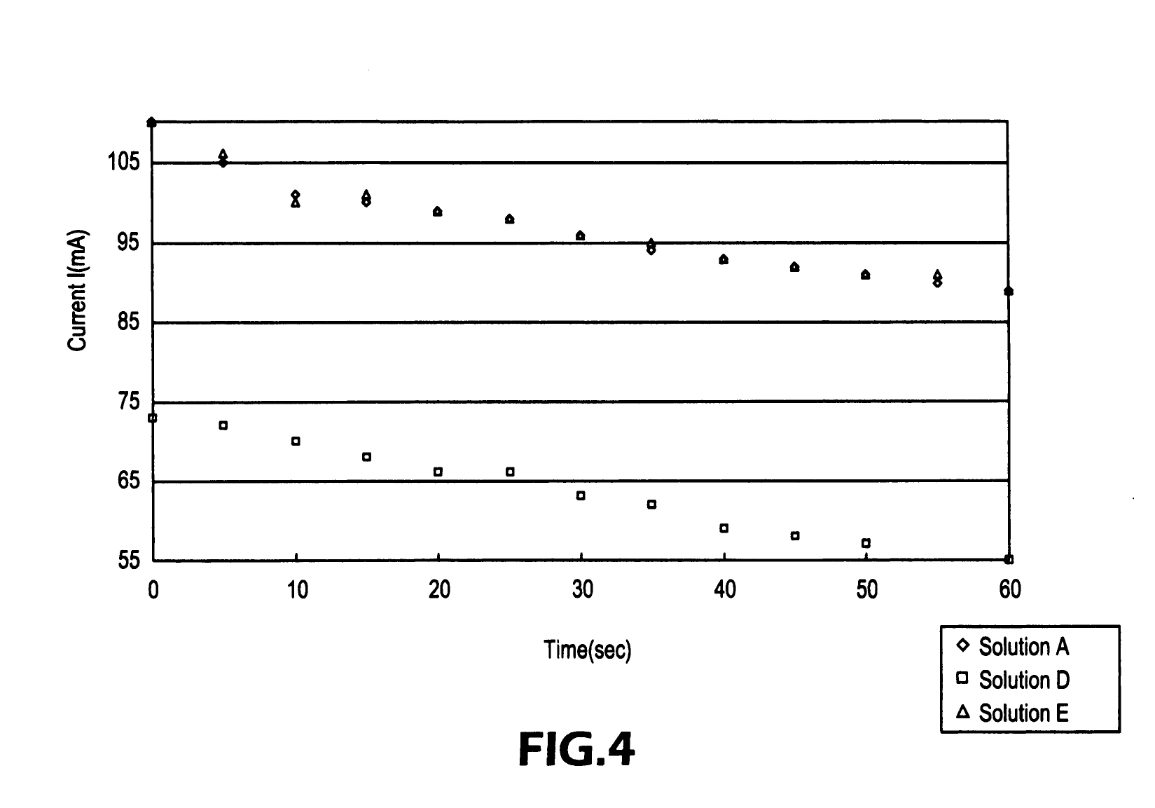

FIG. 5 is a flow chart of the present invention.

### DETAILED DESCRIPTION OF THE INVENTION

The technical characteristics, features and advantages of the present invention will become apparent in the following detailed description of the preferred embodiments with reference to the accompanying drawings.

Since the ion concentration of the solution keeps on changing during the electrophoresis process, therefore the ion concentration and the current conditions of the solution cannot be identified by the information from a single electric appliance. The present invention fixes an electric field and uses the change of current to predict the change of ion concentration in the solution. The actual practice is substantially the same as described below:

If the electrophoresis is performed for several cathode substrates plates under a specific quantity of electrophoresis solution and the time for each electrophoresis deposition is the same, a prefect electrode electron emitter will be deposited. Under such conditions, the current and time for each electrophoresis deposition for each substrate is recorded and the relation of the current per unit time and the time is expressed by the following function.
I=I(t)=α+βt (Equation 1)

Under different charger concentrations, the ratio of coefficients of (α1, β1), (α2, β2) is directly proportional to the concentration. The present invention uses this direct proportion, and the charger concentration falls within a reasonable range and the invention continues to add chargers and carbon nano tube, so as to maintain the solute concentration of the electrophoresis solution at a specific value to provide the best electrophoresis effect. Thus, the present invention can perform the electrophoresis deposition for cathode substrates continuously and achieve the mass production.

In the preliminary stage of the electrophoresis process, if an electric field is added, then the anions and cations of the charger in the solution will be affected by the electric field and deposited onto the two electrodes to constitute the electrophoresis. By then, an obvious change of current occurs. After the surfaces of the electrodes are deposited with ions to a specific quantity or the ion concentration in the solution becomes lesser, the quantity of ion deposition is equal to the quantity of ions, which constitutes an equilibrium state, and thus the change of current becomes mild. Such change is recurrent, and thus the current I per unit time and the electrophoresis time t can be expressed in a function by means of regression as follows:
I=α0+α1t+α2t2+α3t3 (Equation 3)

Where, the high coefficients of the fifth power term is insignificant and can be neglected.

Therefore, the present invention sets the fixed value of an electric field and uses a fixed electric field to carry out the electrophoresis, such that the electrophoresis deposits the charger ions onto the cathode substrate. The anions and cations are deposited gradually onto the electrodes, so that the concentration of the charger ions in the electrophoresis solution drops, and the current also drops accordingly. Since the ammeter on the power supply can detect the intensity of current, therefore the graph of time versus current can be obtained by the solution concentration. The following experiment is taken for example to illustrate the relation between the current and the charger concentration.

The following experiment uses solution A, solution B and solution C as the electrophoresis solutions to carry out the experiment for depositing the carbon nano tube onto the cathode substrate, and ethanol is used as a solvent, and magnesium nitrate (Mg(NO₃)₂) is used as a charger. The carbon nano tubes are added to prepare the three solutions A, B and C with different concentrations of the electrophoresis solution, wherein solution A is an index solution for a general electrophoresis carbon nano tube electron emitter, and the carbon substrates are obtained preferably by the deposition process, and solutions B and solution C simulate a solution. Further, the ratio of the concentrations in percentage by weight of magnesium nitrate and carbon nano tube is a certain specific value in order to obtain the best deposition of carbon nano tubes of the cathode substrate. In the index solution of the present invention, the concentration of magnesium nitrate is 0.025% and the concentration of carbon nano tube is 0.0125%, and the ratio of the concentrations of the magnesium nitrate and carbon nano tube is 2.

In other words,

$$\frac{[ {Mg ( {NO}_{3} )}_{2} ]}{⌈ carbon nano tube ⌉} = 2$$

Solution B and solution C simulate solution A and go through the solution of the electrophoresis, or go through the solution of the electrophoresis for a second time. After the carbon nano tube and magnesium nitrate are deposited, their concentrations drop, and thus the solute concentrations of solution B and solution C are both lower than that of the index solution
A. The electrophoresis conditions adopted by the present invention are given as follows:

Electric Field 10 volt/millimeter

Duty Factor)=⅕(on:total)

Voltage Frequency=250 Hz

With these conditions, the charger concentration falls in the range of 0.020%˜0.03% to obtain the best deposition of the solution.

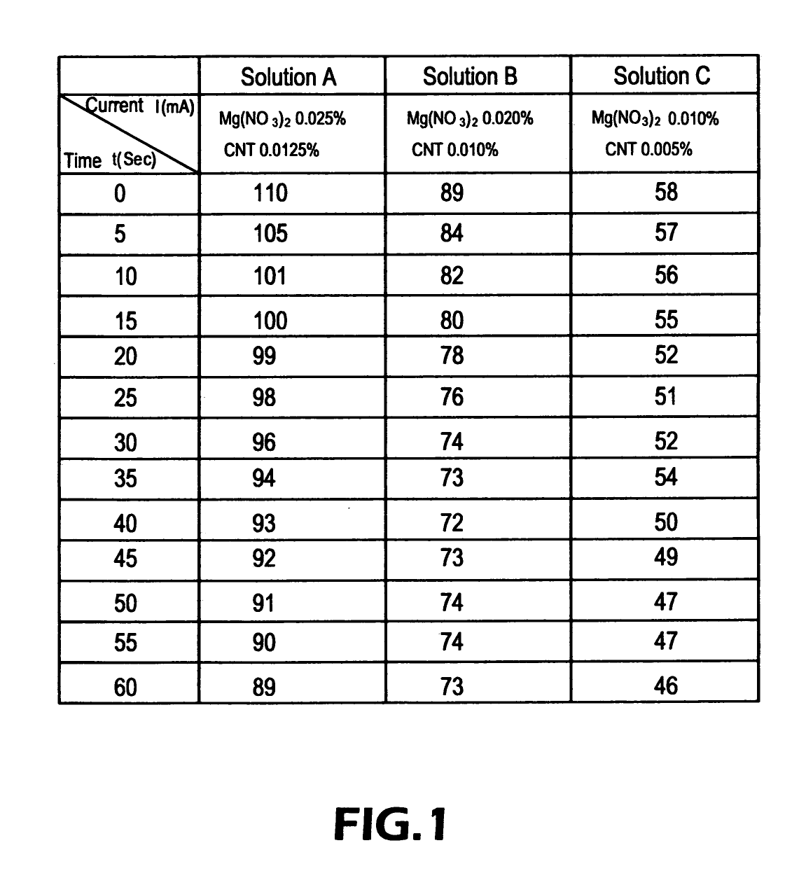

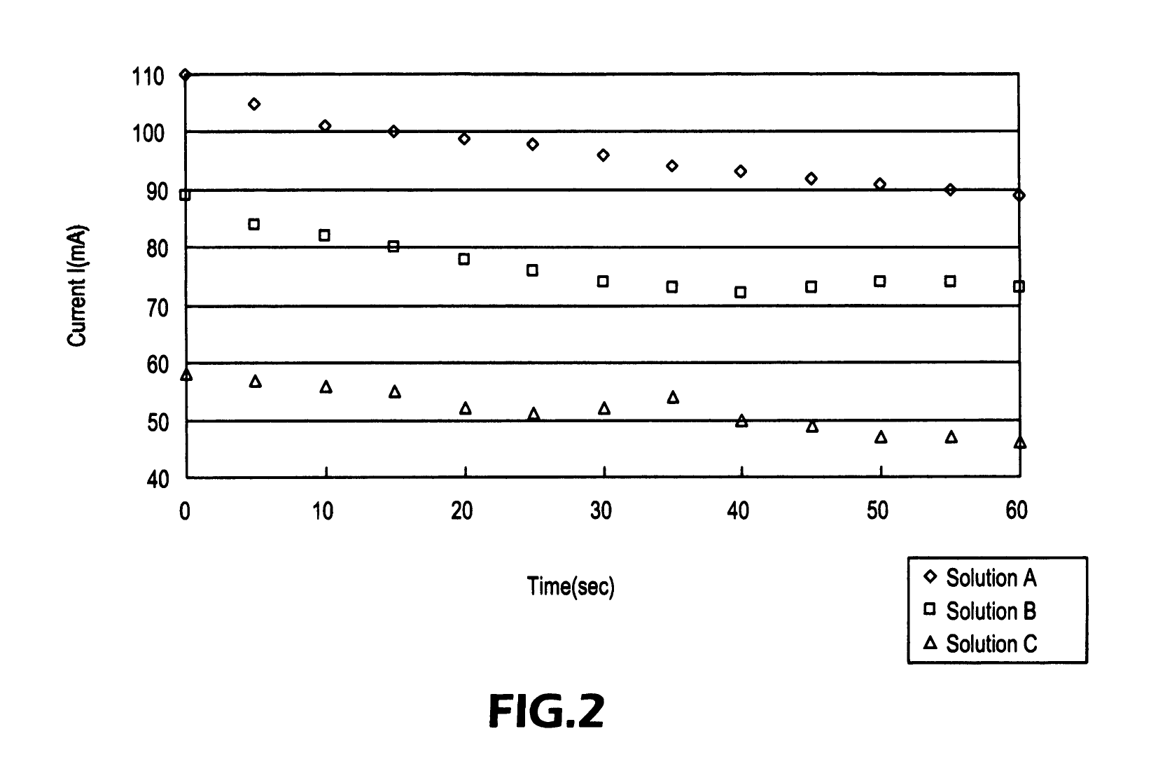

Referring to FIG. 1 for the table showing the relation between the time t and the current I of the three solutions A, B and C after the first minute of the electrophoresis deposition; the unit of current is milliampere (mA); and the unit of time is second (sec). From the data as shown in FIG. 1, a graph of time t versus current I can be obtained. In FIG. 2, the relation between the time t and the current I shows a substantially linear relation.

To further illustrate the linearity between the current I and the time t, the corresponding values of the current I (mA) of an index solution (Solution A) and the electrophoresis time t (sec) are computed by a regression method to obtain a fifth power equation l(t) as follows:
IA(t)=−3×10−7t5+6×10−5t4−0.0036t3+0.1012t2−1.5637t+110.14 (Equation 5 )

Where, the ratios of coefficients of each power term and the linear term are given below (the positive and negative signs are not taken into consideration):

Coefficient of quadric term:Coefficeint of linear term=0.1012:1.5637 ˜6.47×10−2

Coefficient of cubic term:Coefficeint of linear term=3.6×10−3:1.5637˜2.30×10−3

Coefficient of 4^{th} power term:Coefficeint of linear term=6×10−5:1.5637˜3.83×10−5

Coefficient of 5^{th} power term:Coefficeint of linear term=3×10−7:1.5637˜1.92×10−7

In other words, the difference of coefficients between the quadric term and the linear term is two orders, the difference of coefficients between the cubic term and the linear term is three orders the difference of coefficients between the the fourth power term and the linear term is five orders, and the difference of coefficients between the fifth power term and the linear term is seven orders. Therefore, the coefficients of a higher power derived from the equation are insignificant. Since the coefficient of a higher power term has a difference of at least two orders, therefore such terms can be considered as insignificant terms. The relation between the current I and the time t of the index solution (Solution A can be expressed as follows:
IA(t)≈110.14−1.5637t (Equation 6)

Therefore, the current IA and the time t of Solution A have a linear relation.

Similarly, the aforementioned procedure is repeated, and the current I (mA) and the electrophoresis time t (sec) of Solution B also show a linear relation. Firstly, the equation is computed by a regression method to obtain a fifth power equation IB(t) as follows:
IB(t)=5×10−7t5+7×10−5t4−0.0037t3+0.0843t2−1.2493t+88.889 (Equation 7)

Where, the coefficients of the high power terms and the linear term are given below (the positive and negative signs are not taken into consideration):

Coefficient of quadric term:Coefficeint of linear term=0.0843:1.2493≈6.75×10−2;

Coefficient of cubic term:Coefficeint of linear term=0.0037:1.2493≈2.96×10−3;

Coefficient of the 4^{th} term:Coefficeint of linear term=7×10−5:1.2493˜5.60×10−5;

Coefficient of the 5^{th} term:Coefficeint of linear term=5×10−7:1.2493˜4.00×10−7;

In other words, the difference of coefficients of the quadric term, the cubic term, the fourth power term, and the fifth power term with the linear term is at least two orders. The result is the same as that of Solution A, and it is reasonable to assume that the coefficients of the higher power terms are even less significant. Therefore, the relation between the current IB and the time t for Solution B is expressed as follows:
IB(t)≈88.8889−1.2493t (Equation 8)

Thus, the current IB and the time t of Solution B show a linear relation.

In summation of the description above, it is known from Equations 6 and 8 that the electrophoresis time and current substantially show a linear relation under reasonable conditions (with specific ranges of constant electric field, duty factor, and voltage frequency and the solute concentration of the electrophoresis solution falls in a specific range, wherein the concentration of magnesium nitrate is 0.02%˜0.025% and the concentration of carbon nano tube is 0.01%˜0.0125%). In other words,
I(t)=Ε+βt (Equation 1)

It is worthy to note that the ratio of the charger concentrations of Solutions B and A is 0.020 :0.025=0.80, and the coefficients of their constant term and linear term are expressed as follows respectively:

$$\frac{{α}_{B}}{{α}_{A}} = \frac{88.8889}{110.14} \approx 0.81 ;$$

$$\frac{{β}_{B}}{{β}_{A}} = \frac{( - 1.2493 )}{( - 1.5637 )} \approx 0.80 ;$$

Therefore, the ratio of coefficients αA:αB and βA:βA can satisfy the ratio of the concentrations of chargers. In other words,

$$\frac{{α}_{B}}{{α}_{A}} = \frac{{β}_{B}}{{β}_{A}} = \frac{[ Charger ions of Solution B ]}{[ Charger ions of Solution A ]} \qquad ( Equation 9 )$$

Solution C is an electrophoresis solution with low concentrations of chargers and carbon nano tubes, and such solution no longer can carry out the electrophoresis under the currently set electrophoresis conditions. From the data of the current and the time of the solution C, the equation IC (t) can be obtained, and the ratio of coefficients of each term and index solution does not satisfy the relation as shown in Equation 6. Therefore, the ratio of coefficients of IC (t) and IA (t) cannot be used to derive the concentration. The relation as shown in Equation 6 cannot be applied to solution C as follows:

Referring to FIG. 1 for the relation between the current IC and the time t of the solution C, the equation is obtained by regression:
IC(t)=5×10−7t5−8×10−5t4+3.7×10−3t3−0.072t2+0.2031t+57.871 (Equation 10)

Similarly, the coefficients of the higher power terms are insignificant, and thus can be neglected. Thus, the equation can be simplified as follows:
IC(t)≈0.2031t+57.871 (Equation 11)

By then, the ratio of coefficients of solution A and solution C as shown in Equations 2 and 7 respectively can be found.
Ac:αA−57.871:110.14≈0.53;
Bc:βA−0.2031:(−1.5637)≈(−0.13);

Since the ratio of solute concentrations of solution C and solution A is 0.01: 0.025=0.4, the coefficients obtained above are not related with 0.4. In other words,

$$\frac{{α}_{C}}{{α}_{A}} \neq \frac{{β}_{C}}{{β}_{A}} \neq \frac{[ Charger ions of Solution B ]}{[ Charger ions of Solution A ]} . \qquad ( Equation 12 )$$

In summation of the description above, if the charger concentrastion of a solution is 0.03%˜0/.020% under the foregoing experiment conditions, the ratio of coefficients of the current and the time as well as the relation of the index solution will be equal to the charger concentration of a solution and the index solution. As mentioned in Equation 9,

$$\begin{matrix}\frac{{α}_{a solution}}{{α}_{index solution}} = \frac{[ Charger ions of a solution ]}{[ Charger ions of an index solution ]} & ( Equation 13 ) \\ \frac{{β}_{a solution}}{{β}_{index solution}} = \frac{[ Charger ions of a solution ]}{[ Charger ions of an index solution ]} & ( Equation 14 )\end{matrix}$$

In this range, the electrophoresis deposition produces the best cathode substrate and defines an appropriate concentration of electrophoresis. If the concentration of chargers drops below 0.010%, then the relations as shown in Equations 13 and 14 do not exist.

According to the conclusions of Equations 13 and 14, the concentrations of the chargers and the carbon nano tubes in the electrophoresis solution are provided for reference. Assumed that the concentration of chargers falls within a range of 0.03%˜0.020%, the testing solution F can use the data of the time t and the current IF at the prelimiarny stage of the electrophoresis process to compute a linear equation and then uses Equations 13 and 14 to derive the ion concentration of the chargers in solution F. If the difference of

$$\frac{{α}_{a solution}}{{α}_{index solution}} and \frac{{β}_{a solution}}{{β}_{index solution}}$$

exceeds 5%, then it means that the solute concentration drops beyond the appropriate range of concentration, and thus we need Equation 13 to find the concentration and resupply the solutes.

Further, the intensity of current and the table of the data of the electrophoresis time are recursive, and thus the data in the table can be used for determining and resupplying the chargers and carbon nano tubes to maintain a fixed quantity effect, so that the electrophoresis deposition is applicable for the deposition process for mass production. The present invention uses the table of the time and the current to determine whether or not the concentration of the present chargers and carbon nano tubes in the electrophoresis solution is appropriate and also determine whether the solute concentration of the present electrophoresis solution requires a resupply of solutes for controlling the electrophoresis process to obtain the best effect of depositing carbon nano tubes.

A preferred embodiment is adopted according to the actual needs, and the detailed operating procedure and the obtained data are used together with the table for the detailed description of the present invention.

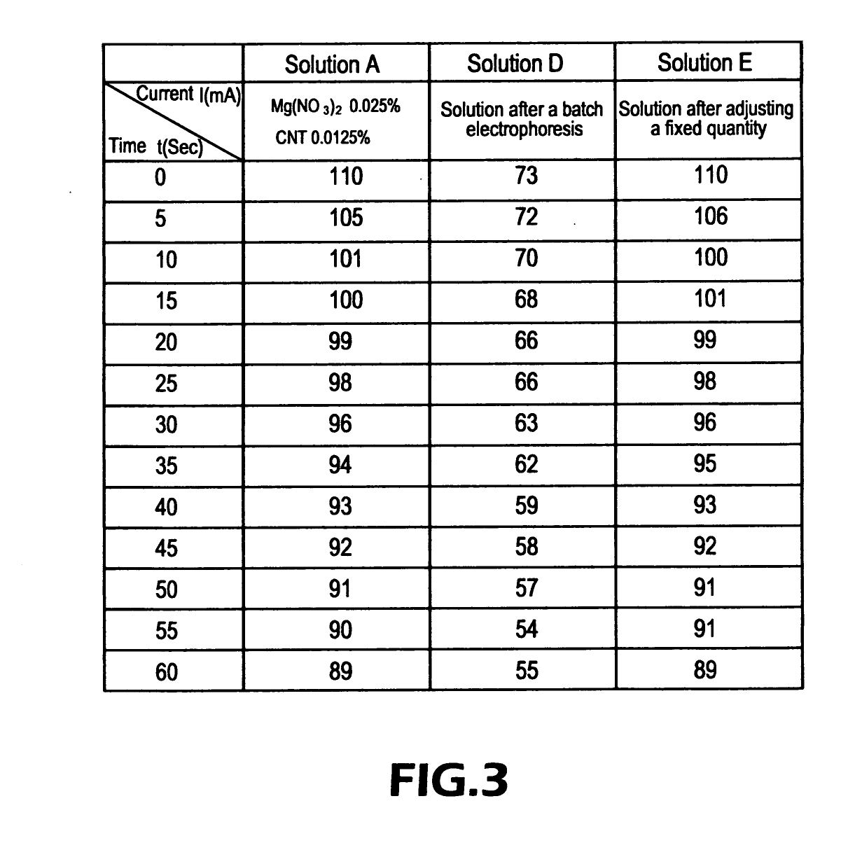

After solution A has gone through the electrophoresis for several times to obtain solution D. In the meantime, the corresponding current and time for solution A and solution D are recorded in the table as shown in FIG. 3. The equation of the current and the time of solution D is given as follows:
ID(t)=2×10−7t5−2×10−5t4+0.0011t3−0.0216t2−0.1809t+0.1809t+73.086≈−0.1809 t+73.086

Where, the ratio of coefficients of the linear terms is 1.51 and the ratio of coefficients of the constant term is 8.64, and their difference is 82% as computed below, and thus the solute concentration drops below 0.02%, and the elecrophoresis process requires resupplying the chargers and carbon nano tube ions.

$$\begin{matrix}\vert \frac{\begin{matrix}Coefficient of linear term - \\ Coefficient of constant\end{matrix}}{Coefficient of constant} \vert = \vert \frac{1.51 - 8.64}{8.64} \vert \\ = 82 \% > 5 \%\end{matrix}$$

Then, the solute concentration of solution D can be derived as follows. From Equation 13,

$$\frac{73.086}{110.14} = \frac{[ Charger Ions of Solution D ]}{0.025 \%}$$

Therefore, the concentration of charger ions in solution A is 0.0166%, and the concentration of the solution is increased to the concentration of the index solution, and thus it is necessary to resupply 0.0084% of chargers and 0.0042% of carbon nano tubes in solution D to compensate the electrophoresis of solution E.

To make sure that the resupply for solution E meets the standard of the concentration for the index solution, the relation of current and time of solution E as shown in IE(t) is used for the determination.
IE(t)=4×10−7t+6×10−5t4−0.0037t3+0.1046t2−1.588t+110.36≈−1.588t+110.36

Where, the ratio of coefficients of the constant term and the ratio of coefficients of the linear term are computed as follows. Since their values are approximately equal to 1, therefore it is reasonable to predict that the concentration of the solution E meets the standard of concentration of the index solution.

The ratio of coefficients of the constant terms is

$$\frac{{α}_{E}}{{α}_{A}} = \frac{110.36}{110.14} \approx 1 ;$$

The ratio of coefficients of the linear terms is

$$\frac{{β}_{B}}{{β}_{A}} = \frac{( - 1. .588 )}{( - 1.5637 )} \approx 1 ;$$

In other words, the difference of the computed results

$$\frac{{α}_{D}}{{α}_{A}} and \frac{{β}_{D}}{{β}_{A}}$$

is too large. Although we can process with the electrophoresis, a larger voltage or a longer time is needed or more times of electrophoresis is required to meet the requirements of the deposition effect. Therefore, the inventor of the present invention adds the carbon nano tubes and chargers in a specific ratio into the solution D, which approaches the standard liquid for carrying out the electrophoresis deposition for the next time. The obtained data is the linear data as shown in FIG. 3 and the difference between

$$\frac{{α}_{E}}{{α}_{A}} and \frac{{β}_{E}}{{β}_{A}}$$

falls within a range of 5%, and the electrophoresis deposition effect also meets the requirements of the sample.

In summation of the description above, the present invention carries out an electrophoresis operated at a fixed electric field and expresses the current between the electrodes and the time by a polynomial. In other words, the current I is expressed as a function of time t which is I=I(t). The ratio of coefficients is used for determining the concentration of the electrophoresis solution. The method of the present invention is illustrated by the description accompanied with the flow chart as follows:

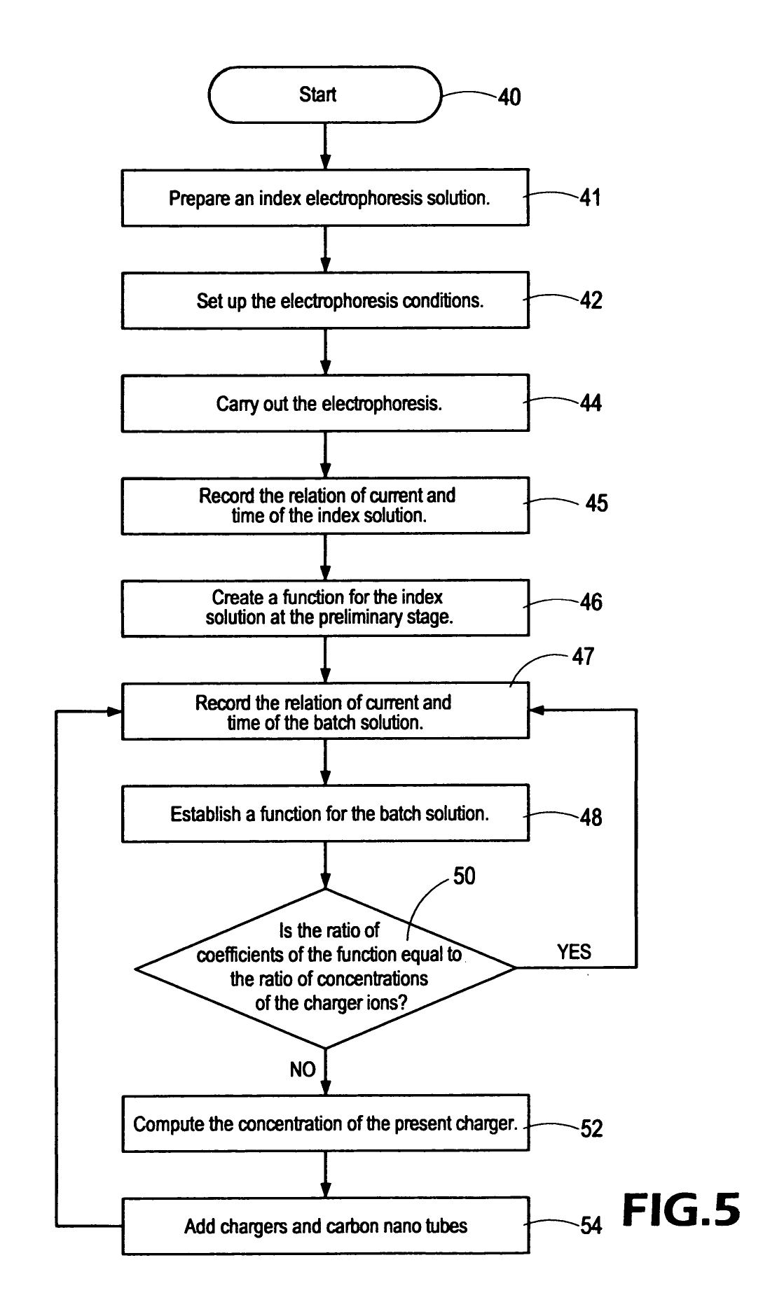

Referring to FIG. 5 for the flow chart of the present invention, it is necessary to prepare an index electrophoresis solution first and set up the electrophoresis conditions as shown in Steps 40 to 42. The index electrophoresis solution is a solution of the best cathode substrate obtained from the electrophoresis deposition. In this invention, the concentration of magnesium nitrate in the index solution is 0.025% and the concentration of carbon nano tubes in the index solution is 0.0125%, and the ratio of concentrations of the index solution and the carbon nano tubes is equal to 2. In the meantime, the present invention adopts a fixed electric field for the electrophoresis, and the electric field=10 volts/milliampere, and the duty factor is set to ⅕(on/total), and the voltage frequency is equal to 250 Hz. In Step 44, the electrophoresis is carried for the index solution under the foregoing electrophoresis conditions. In the meantime, the corresponding data of the current per unit time and the time at the preliminary stage of the electrophoresis are used for the regression and expressing the current in terms of a function of time which is Ii=Ii(t). In a preferred embodiment, the unit time is 60 seconds, and the current is recorded once every five seconds.

Then, the batch electrophoresis is continued for the solution, and similarly the same unit time is used for establishing the function Ib=Ib(t) of the electrophoresis solution as shown in Steps 47 and 48. In Step 50, the ratio between the linear terms and constant terms of Ib(t) and Ii(t) is used to predict the limited use of ion concentration in the solution. If difference of the ratio of coefficients of the constant term and the linear term exceeds a critical value, it means that the concentration of chargers is lower than the appropriate concentration. In Step 54, the concentration of charges of the present solution is computed, and the carbon nano tubes and chargers are resupplied, so as to approach the index solution and facilitate the electrophoresis for the next time.

In this method, the electrophoresis deposition is adopted for preparing the cathode substrate of the electron emitter, and a continuous batch electrophoresis is carried out to predict and know about the change of ion concentration in the solution, and a fixed quantity is adjusted and resupplied to meet the requirement of a linear relation, so that the electrophoresis can be carried out in a proper sequence.

The present invention are illustrated with reference to the preferred embodiment and not intended to limit the patent scope of the present invention. Various substitutions and modifications have suggested in the foregoing description, and other will occur to those of ordinary skill in the art. Therefore, all such substitutions and modifications are intended to be embraced within the scope of the invention as defined in the appended claims.

What is claimed is:

1. A method of determining and controlling concentration of an electrophoresis solution of a field emission display cathode substrate carbon nano tube, comprising the steps of:
(a) adding carbon nano tubes in a solvent and assisting an ion to prepare an index electrophoresis solution, and setting up the conditions for an electrophoresis;
(b) performing the electrophoresis to the index electrophoresis conditions, under the set electrophoresis conditions;
(c) obtaining the data of corresponding current per unit time and the time in an electrophoresis at a preliminary stage;
(d) expressing the current of step (c) in terms of a function Ii of time;
(e) continuing the batch electrophoresis for the solution;
(f) establishing a function Ib of the current and the time of the batch electrophoresis solution per unit time;
(g) comparing the ratio of the coefficients of a linear term and a constant of the Ib(t) and Ii(t);
(h) computing the charger concentration of the batch solution, if the difference between the two values exceeds a critical error, resupply the carbon nano tubes and the chargers.

2. The method of claim 1, wherein the index electrophoresis solution deposits an appropriate carbon nano tube solution at two electrodes under the electrophoresis conditions.

3. The method of claim 1, wherein the charger ion of the index solution is magnesium nitrate.

4. The method of claim 3, wherein the magnesium nitrate has a concentration substantially falling in the range of 0.030%˜0.02% by weight.

5. The method of claim 4, wherein the carbon nano tube has a concentration substantially falling in the range of 0.0155%˜0.01% by weight.

6. The method of claim 3, wherein the ratio of the concentrations by weight of the charger and the carbon nano tube is substantially equal to 2.

7. The method of claim 1, wherein the electrophoresis conditions include performing the electrophoresis under a fixed electric field.

8. The method of claim 7, wherein the electric field is equal to 10 volt per millimeter.

9. The method of claim 7, wherein the electrophoresis conditions include a duty factor of the power supply equal to ⅕ on/total.

10. The method of claim 7, wherein the electrophoresis conditions include a voltage frequency of the power supply equal to 250 Hz.

11. The method of claim 1, wherein the unit time is equal to 60 seconds.

12. The method of claim 1, wherein the current is recorded once every 5 minutes.

13. The method of claim 1, wherein the critical error is equal to 5%.

14. The method of claim 1, wherein the solution uses an organic solvent as a solvent.

15. The method of claim 14, wherein the solution uses ethanol as a solvent.
